(19) United States
(12) Patent Application Publication
(10) Pub. No.: US 2021/0236398 A1
(43) Pub. Date: Aug. 5, 2021

(54) COMPOSITIONS AND METHODS FOR HAIR

(71) Applicant: L'OREAL, Paris (FR)

(72) Inventors: Rachel FEREBEE, Morristown, NJ (US); Guojin ZHANG, Westfield, NJ (US); Ronak RUGHANI, Edison, NJ (US)

(21) Appl. No.: 17/151,571

(22) Filed: Jan. 18, 2021

Related U.S. Application Data
(60) Provisional application No. 62/968,854, filed on Jan. 31, 2020.

(30) Foreign Application Priority Data
Mar. 5, 2020 (FR) 2002231

(51) Int. Cl. A61K 8/24; A61Q 5/00; A61K 8/55; A61K 8/64; A61K 8/44

(52) U.S. Cl. CPC A61K 8/24; A61Q 5/00; A61K 8/44; A61K 8/64; A61K 8/55

(57) ABSTRACT

The disclosure relates to compositions and methods for treating hair, protecting hair, and/or reducing damage to hair. The compositions comprise at least one compound comprising at least one phosphate group. The methods comprise applying the compositions to hair.

### TECHNICAL FIELD

The present disclosure relates to compositions and methods for treating hair, protecting hair, or reducing damage to hair.

### BACKGROUND

Individuals desire healthy and strong hair, as healthy-looking hair is in general considered to be a sign of good health and good hair-care practices. However, nutrition, environmental influences, heat, and chemical hair treatments can lead to hair damage that significantly weakens and dulls the hair over time. In particular, conventional styling processes may be damaging to hair, such as heat-styling hair with heat tools (e.g. flat iron, curling iron) or chemical styling treatments (e.g. perming, relaxing). Hair that has been weakened or damages is also prone to be frizzy, to have breakage, and to the formation of “split ends.”

Thus, there is a need for compositions and methods for protecting hair, wherein the compositions have desired cosmetic benefits and composition properties that protect hair from damage or reduce hair damage, such as heat-induced damage.

### SUMMARY

It has surprisingly been found that compositions according to the disclosure protect hair from damage or reduce hair damage from heat treatments, such as heat treatments with conventional heat tools (e.g. flat iron, curling iron). The hair protecting composition may comprise at least one compound comprising at least one phosphate group, wherein the pH of the composition is optionally less than or equal to about 7.5.

The at least one compound comprising at least one phosphate group may be present in an amount of up to about 10%, such as up to about 5%, such as ranging from about 0.001% to about 10%, about 0.1% to about 5%, or about 0.2% to about 2%, based on the total weight of the composition.

The at least one compound having at least one phosphate group may be chosen from monosodium phosphate, phytic acid or glycerol phosphate, or combinations thereof. In a further embodiment, the at least one compound having at least one phosphate group is phytic acid. In a further embodiment, the at least one compound having at least one phosphate group is glycerol phosphate. In a further embodiment, the at least one compound having at least one phosphate group is monosodium phosphate. When the at least one phosphate group is phytic acid, the phytic acid may be present in an amount ranging from about 0.001% to about 10%, relative to the total weight of the composition. When the at least one phosphate group is monosodium phosphate, the monosodium phosphate may be present in an amount ranging from about 0.001% to about 2%, relative to the total weight of the composition. When the at least one phosphate group is glycerol phosphate, the glycerol phosphate may be present in an amount ranging from about 0.001% to about 10%, relative to the total weight of the composition.

In an embodiment, the composition optionally comprises at least one amino acid, at least one peptide oligomer, at least one protein oligomer, at least one hydrolyzed protein, or combinations thereof. A separate composition which comprises at least one amino acid, at least one peptide oligomer, at least one protein oligomer, at least one hydrolyzed protein, or combinations thereof may also be used prior to, simultaneously with, or after the composition comprising at least one compound comprising at least one phosphate group.

The at least one amino acid, at least one hydrolyzed protein, at least one peptide oligomer, at least one protein oligomer, or combination thereof may be present in an amount of up to about 5%, or up to about 1%, based on the total weight of the composition.

In a further embodiment, the hair protecting composition comprises: at least one compound having at least one phosphate group chosen from: a. phytic acid, wherein the phytic acid is present in an amount ranging from about 4.5% to about 0.2%, relative to the total weight of the composition, b. glycerol phosphate, wherein the glycerol phosphate is present in an amount ranging from about 2% to about 0.2%, relative to the total weight of the composition, or c. monosodium phosphate, wherein the monosodium phosphate is present in an amount ranging from about 2% to about 0.2%, relative to the total weight of the composition. In a further embodiment, the hair protecting composition further comprises at least one amino acid, at least one hydrolyzed protein, at least one peptide oligomer, at least one protein oligomer, or combination thereof present in an amount of up to about 1%, based on the total weight of the composition.

Methods of protecting hair or reducing heat-induced damage to hair according to the disclosure may comprise applying to the hair a composition comprising at least one compound comprising at least one phosphate, wherein the pH of the composition is optionally less than or equal to about 7.5.

Further methods of protecting hair or reducing heat-induced damage to hair according to the disclosure may comprise: (1) applying to the hair a first composition comprising at least one amino acid, at least one hydrolyzed protein, at least one peptide oligomer, at least one protein oligomer, or combination thereof, and (2) applying to the hair a second composition comprising at least one compound comprising at least one phosphate. In various embodiments, the first composition may be applied to hair before or after the second composition; the first composition and second composition may be applied individually but simultaneously; or the first composition and second composition may be mixed prior to applying the compositions to the hair.

The methods may optionally further comprise a second step comprising heating the hair at a temperature of about 40° C. to about 210° C., wherein said second step occurs after applying to the hair a hair protecting composition. In a further embodiment, the method may comprise blow-drying the hair after application of the composition(s). In a further embodiment, the method may further comprise straightening or curling the hair with heat after application of the composition(s). In a further embodiment, the method may comprise blow-drying the hair and straightening or curling the hair with a flat iron or curling iron after application of the composition(s).

In another embodiment, methods of protecting hair or reducing heat-induced damage to hair according to the disclosure may comprise: (1) applying to the hair a first composition comprising at least one compound comprising at least one phosphate, (2) applying to the hair a second composition comprising at least one amino acid, at least one hydrolyzed protein, at least one peptide oligomer, at least one protein oligomer, or a combination thereof, and (3) heating the hair at a temperature of about 40° C. to about 210° C. after application of the first and second compositions.

### BRIEF DESCRIPTION OF THE DRAWINGS

Implementation of the present technology will now be described, by way of example only, with reference to the attached figures, wherein:

FIG. 1 compares the Denaturation temperature (Td) of swatches of hair treated with the hair protecting compositions of the present disclosure.

FIG. 2 compares the Denaturation temperature (Td) of swatches of hair treated with the hair protecting compositions of the present disclosure.

FIG. 3 compares the Denaturation temperature (Td) of swatches of hair treated with the hair protecting compositions of the present disclosure.

FIG. 4 compares the Denaturation temperature (Td) of swatches of hair treated with the hair protecting compositions of the present disclosure.

FIG. 5 compares the Denaturation temperature (Td) of swatches of hair treated with the hair protecting compositions of the present disclosure.

FIG. 6 compares the Denaturation temperature (Td) of swatches of hair treated with the hair protecting compositions of the present disclosure.

FIG. 7 compares the Denaturation temperature (Td) of swatches of hair treated with the hair protecting compositions of the present disclosure.

FIG. 8 compares the Denaturation temperature (Td) of swatches of hair treated with the hair protecting compositions of the present disclosure.

It should be understood that the various aspects are not limited to the arrangements and instrumentality shown in the drawings.

### DESCRIPTION

The disclosure relates to compositions and methods for protecting hair. The compositions comprise phosphate-containing compounds, or synergistic combinations of a) phosphate-containing compounds and b) amino acids, hydrolyzed protein, peptide oligomers, or combinations thereof. The methods comprise applying the compositions to the hair.

### I. Compositions

The compositions according to the disclosure comprise at least one at least one compound having at least one phosphate group, or combinations of a) at least one compound having at least one phosphate group and b) at least one amino acid, hydrolyzed protein, peptide oligomer, or combinations thereof.

### Phosphates

Compositions according to the disclosure comprise at least one compound having at least one phosphate group, also referred to as a phosphate-containing compound. Exemplary and non-limiting phosphate compounds that can be used include: alkali metal phosphates such as potassium phosphate, potassium dihydrogen phosphate, dipotassium hydrogen phosphate, sodium phosphate (also referred to as “monosodium phosphate” or “MSP”), sodium dihydrogen phosphate, disodium hydrogen phosphate, calcium phosphate, magnesium phosphate, zinc phosphate; glycerol phosphate (or “glyceryl phosphate” or “glycerophosphate”), phytic acid, as well as combinations thereof. Glycerol or glyceryl phosphate may be available as a metal phosphate such as disodium glycerol phosphate (or sodium glycerol phosphate and commercially available from the company SEPPIC), calcium glycerol phosphate, magnesium glycerol phosphate, disodium diglyceryl monophosphate, or aluminum isostearyl glyceryl phosphate.

Polyphosphorus derivatives may be chosen from inorganic polyphosphates, preferably chosen from: optionally hydrated alkali metal pyrophosphates, preferably chosen from sodium pyrophosphate, potassium pyrophosphate, sodium pyrophosphate decahydrate; and polymetaphosphates, such as sodium hexametaphosphate, sodium trimetaphosphate; sodium polyphosphates such as sodium tripolyphosphate, and mixtures thereof. Sodium and/or potassium is preferably used as alkali metal. According to another embodiment, polyphosphorus derivatives are chosen from organic polyphosphorus derivatives, preferably from polyphosphoric acids and/or salts thereof, such as phytic acid, polyphosphonic acids and/or salts thereof, such as EDTMP, DETMP, ATMP, HEDP, DTPMP, and mixtures thereof.

The total amount of the at least one compound having at least one phosphate group may vary, but typically is up to about 10%, up to about 9%, up to about 8%, up to about 7%, up to about 6%, up to about 5.5%, up to about 5%, up to about 4.5%, up to about 4%, up to about 3.5%, up to about 3%, up to about 2.5%, up to about 2%, up to about 1.5%, up to about 1%, or up to about 0.5%, based on the total weight of the composition. The total amount of the at least one compound having at least one phosphate group may range from about 0.001% to about 10%, about 0.01% to about 5%, about 0.4% to about 4.5%, or about 0.2% to about 2%, based on the total weight of the composition. For example, the total amount of the at least one compound having at least one phosphate group may range from about 0.01% to about 10%, such as from about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, about 0.01% to about 0.75%, about 0.01% to about 0.5%, about 0.05% to about 10%, about 0.05% to about 9%, about 0.05% to about 8%, about 0.05% to about 7%, about 0.05% to about 6%, about 0.05% to about 5%, about 0.05% to about 4%, about 0.05% to about 3%, about 0.05% to about 2%, about 0.05% to about 1%, about 0.05% to about 0.75%, about 0.05% to about 0.5%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.1% to about 0.75%, about 0.1% to about 0.5%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, or about 5% to about 6%, including all ranges and sub-ranges there between, based on the total weight of the composition. In various embodiments, the at least one compound having at least one phosphate group may be present in an amount of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10%, based on the total weight of the composition.

When the at least one compound having at least one phosphate group in the compositions according to the disclosure includes monosodium phosphate, the total amount of monosodium phosphate may range from about 0.01% to about 10%, such as from about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, about 0.01% to about 0.75%, about 0.01% to about 0.5%, about 0.4% to about 10%, about 0.4% to about 9%, about 0.4% to about 8%, about 0.4% to about 7%, about 0.4% to about 6%, about 0.4% to about 5%, about 0.4% to about 4%, about 0.4% to about 3%, about 0.4% to about 2%, about 0.4% to about 1.5%, about 0.4% to about 1.4%, about 0.4% to about 1.2%, about 0.4% to about 1%, about 0.4% to about 0.8%, 0.001% to about 2%, such as from about 0.001% to about 1.5%, about 0.001% to about 1%, about 0.001% to about 0.75%, about 0.001% to about 0.5%, about 0.001% to about 0.4%, about 0.001% to about 0.3%, about 0.001% to about 0.2%, about 0.001% to about 0.1%, about 0.001% to about 0.09%, about 0.001% to about 0.08%, about 0.001% to about 0.07%, about 0.001% to about 0.06%, about 0.001% to about 0.05%, relative to the total weight of the composition, including ranges and sub-ranges there between.

When the at least one compound having at least one phosphate group in the compositions according to the disclosure includes phytic acid, the total amount of phytic acid may range from about 0.001% to about 10%, such as from about 0.001% to about 9%, about 0.001% to about 8%, about 0.001% to about 7%, about 0.001% to about 6%, about 0.001% to about 5%, about 0.001% to about 4%, about 0.001% to about 3%, about 0.001% to about 2%, about 0.001% to about 1%, about 0.05% to about 10%, about 0.05% to about 9%, about 0.05% to about 8%, about 0.05% to about 7%, about 0.05% to about 6%, about 0.05% to about 5%, about 0.05% to about 4%, about 0.05% to about 3%, about 0.05% to about 2%, about 0.05% to about 1%, about 0.05% to about 0.75%, about 0.05% to about 0.5%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.1% to about 0.75%, about 0.1% to about 0.5%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, or about 5% to about 6%, including all ranges and sub-ranges there between, relative to the total weight of the composition, relative to the total weight of the composition, including ranges and sub-ranges there between.

When the at least one compound having at least one phosphate group in the compositions according to the disclosure includes glycerol phosphate, the total amount of glycerol phosphate may range from about 0.001% to about 10%, such as from about 0.001% to about 9%, about 0.001% to about 8%, about 0.001% to about 7%, about 0.001% to about 6%, about 0.001% to about 5%, about 0.001% to about 4%, about 0.001% to about 3%, about 0.001% to about 2%, about 0.001% to about 1%, about 0.05% to about 10%, about 0.05% to about 9%, about 0.05% to about 8%, about 0.05% to about 7%, about 0.05% to about 6%, about 0.05% to about 5%, about 0.05% to about 4%, about 0.05% to about 3%, about 0.05% to about 2%, about 0.05% to about 1%, about 0.05% to about 0.75%, about 0.05% to about 0.5%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.1% to about 0.75%, about 0.1% to about 0.5%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, or about 5% to about 6%, including all ranges and sub-ranges there between, relative to the total weight of the composition, relative to the total weight of the composition, including ranges and sub-ranges there between.

In certain embodiments, it may be desirable to choose the amount of the at least one phosphate-containing compound in order to provide a particular molar concentration of the phosphate-containing compound, or a particular concentration of phosphate functional groups.

### pH Adjusters

It may, in at least certain embodiments, be beneficial to adjust the pH of the compositions. By way of example only, the pH of the disclosed compositions may advantageously be at or below a pH of about 7.5.

Compositions according to the disclosure may thus optionally also contain acid and alkali pH adjusters (also modifiers). Such pH adjusters include, but are not limited to, sodium hydroxide, hydrochloric acid, lactic acid, sodium metasilicate, silicate compounds, citric acid, ascorbic acid, and carbonate compounds. The disclosed compositions may also be substantially free of acid and alkali pH adjusters.

Non-limiting examples of acidic pH modifiers include organic acids and/or inorganic acids. In some instances, the compositions include at least one non-polymeric mono, di, and/or tricarboxylic acid. A non-polymeric mono, di, and/or tricarboxylic acid is an organic compound having one (mono), two (di), or three (tri) carboxylic acid groups (—COOH). The non-polymeric mono, di, and tricarboxylic acids, and/or salts thereof, typically have a molecular weight of less than about 500 g/mol, less than about 400 g/mol, or less than about 300 g/mol.

Non-limiting examples of monocarboxylic acids, or salts thereof, include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, salts thereof, and a mixture thereof. In some cases, the hair-treatment compositions include at least lactic acid and/or acetic acid, and/or salts thereof.

Non-limiting examples of dicarboxylic acids and/or salts thereof include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, salts thereof, and a mixture thereof. In some cases, the hair-treatment compositions include oxalic acid, malonic acid, malic acid, maleic acid, salts thereof, or a mixture thereof. Non-limiting examples of tricarboxylic acids and salts thereof include citric acid, isocitric acid, aconitric acid, propane-1,2,3-tricarboxylic acid, benzene-1,3,5-tricarboxylic acid, salts thereof, and mixtures thereof. In some instances, the hair-treatment compositions include at least citric acid and/or salts thereof.

In some instances, the hair-treatment composition may include one or more inorganic pH modifiers. Non-limiting examples include hydrochloric acid, phosphoric acid, sulfuric acid, hydrobromic acid, hydriodic acid, phosphoric acid, boric acid, etc.

### Amino Acids, Hydrolyzed Proteins and Peptide Oligomers

Compositions according to the disclosure may optionally also contain at least one amino acid, at least one hydrolyzed protein, at least one “peptide” or “protein” oligomer, or combinations thereof. Without wishing to be bound by theory, it is believed that exogenous amino acids, hydrolyzed proteins, and peptide or protein oligomers may have charge interactions with the phosphate-containing compounds in the composition, providing further heat protection benefits.

Exemplary and non-limiting amino acids that can be used include lysine, threonine, aspartic acid, glutamic acid, arginine, serine, alanine, glycine, isoleucine, leucine, valine, or mixtures thereof.

Non-limiting examples of hydrolyzed protein include hydrolyzed soy protein, hydrolyzed rice protein, hydrolyzed oat protein, hydrolyzed wheat protein, hydrolyzed vegetable protein, hydrolyzed pea protein, hydrolyzed corn protein, etc.

Peptide oligomers may comprise at least one repeating amino acid residue. The repeating amino acid residue can be glycine, L-alanine, L-valine, L-leucine, L-isoleucine, L-proline, L-methionine, L-phenylalanine, L-tryptophan, L-serine, L-threonine, L-cysteine, L-tyrosine.

In certain embodiments, the compositions comprise at least one amino acid, such as two or more amino acids. In further embodiments, the compositions comprise at least one hydrolyzed protein, such as two or more hydrolyzed proteins. In yet further embodiments, the compositions comprise at least one peptide or protein oligomer, such as two or more peptide or protein oligomers. In yet further embodiments, the compositions comprise a combination of at least one amino acid, at least one hydrolyzed protein, and/or at least one peptide or protein oligomer.

The total amount of the at least one amino acid, at least one hydrolyzed protein, at least one peptide or protein oligomer, or combinations thereof may vary, but in various embodiments may be up to about 10%, such as up to about 5%, up to about 4%, up to about 3%, up to about 2%, up to about 1%, or up to about 0.5%, based on the total weight of the composition, including ranges and sub-ranges there between. The amount of the at least one amino acid, at least one hydrolyzed protein, at least one peptide or protein oligomer, or combinations thereof may range from about 0.001% to about 10%, such as from about 0.01% to about 5%, about 0.05% to about 3%, about 0.1% to about 2%, or about 0.5% to about 1%, based on the total weight of the composition, including ranges and sub-ranges there between. For example, the total amount of the at least one amino acid, at least one hydrolyzed protein, at least one peptide or protein oligomer, or combinations thereof may range from about 0.01% to about 10%, about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1.5%, or about 0.5% to about 1%, including all ranges and sub-ranges there between, based on the total weight of the composition, including ranges and sub-ranges there between. In certain embodiments, the at least one amino acid, at least one hydrolyzed protein, at least one peptide or protein oligomer, or combinations thereof may be present in an amount of about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2%, based on the total weight of the composition, including ranges and sub-ranges there between.

Optionally, the weight ratio of the total amount of a) the at least one compound having at least one phosphate group to b) the at least one amino acid, at least one hydrolyzed protein, at least one peptide or protein oligomer, or combinations thereof may be chosen to range from about 1:100 to about 100:1, from about 1:50 to about 50:1, from about 1:25 to about 25:1, from about 1:15 to about 15:1, from about 1:10 to about 10:1, from about 1:9 to about 9:1, from about 1:8 to about 8:1, from about 1:7 to about 7:1, from about 1:6 to about 6:1, from about 1:5 to about 5:1, from about 1:4 to about 4:1, or from about 1:3 to about 3:1, from about 1:2 to about 2:1, from about 1:1 to about 1:1, from about 1:1 to about 1:0.5, from about 1:0.9 to about 1:0.6, from about 1:0.9 to about 1:0.7, including ranges and sub-ranges there between. Optionally, the weight ratio of the total amount of a) the at least one compound having at least one phosphate group to b) the at least one amino acid, at least one hydrolyzed protein, at least one peptide or protein oligomer, or combinations thereof may be about 1:100, about 1:50, about 1:25, about 1:15, about 1:10, about 1:9, about 1:8, about 1:7, about 1:6, about 1:5, about 1:4, about 1:3, about 1:2, about 1:1, about 1:0.9, about 1:0.8, about 1:0.7, about 1:0.5, about 100:1, about 50:1, about 25:1, about 15:1, about 10:1, about 9:1, about 8:1, about 7:1, about 6:1, about 5:1, about 4:1, about 3:1, about 2:1, about 0.9:1, about 0.8:1, about 0.7:1, or about 0.5:1, including ranges and sub-ranges there between.

### Solvents

In certain exemplary embodiments, compositions according to the disclosure comprise at least one physiologically acceptable medium. The physiologically acceptable medium may be chosen from water or a mixture of water and at least one cosmetically acceptable solvent. Non-limiting examples of cosmetically acceptable solvents include C2-C6 lower alcohols, such as ethanol and isopropanol; polyols, especially those containing from 2 to 6 carbon atoms, for instance glycerol, propylene glycol, butylene glycol, pentylene glycol, hexylene glycol, dipropylene glycol or diethylene glycol; polyol ethers, for instance 2-butoxyethanol, propylene glycol monomethyl ether and diethylene glycol monomethyl ether or monoethyl ether; aromatic alcohols such as benzyl alcohol; and mixtures thereof.

In one exemplary embodiment, the composition comprises a solvent or solvent mixture in an amount up to about 99.9%, such as ranging from about 50% to about 99%, about 55% to about 95%, or about 60% to about 90%, by weight relative to the weight of the composition. For example, the composition may comprise water in an amount up to about 99%, such as, for example, an amount ranging from about 50 to about 95%, or from about 60% to about 90%, by weight relative to the weight of the composition.

### Additional Components

The compositions according to the disclosure may optionally also comprise additives useful or desired for preparing hair protecting compositions. For example, it may be beneficial to include components that will aid in application of the composition, such as glycerol. Exemplary and non-limiting additives include nacreous agents, dyes or pigments, fragrances, mineral, plant or synthetic oils, waxes, vitamins, proteins including ceramides, vitamins, UV-screening agents, free-radical scavengers, antidandruff agents, hair-loss counteractants, hair restorers, preserving agents, and mixtures thereof. A person skilled in the art will take care to select the optional additives and the amount thereof such that they do not harm the properties of the compositions of the present disclosure.

The compositions of certain embodiments may comprise stabilizers, for example sodium chloride, magnesium dichloride or magnesium sulfate.

The compositions according to the disclosure may additionally comprise cosmetic adjuvants chosen from fragrances, pigments, chelating agents, softeners, antioxidants, opacifiers, stabilizers, moisturizing agents, vitamins, bactericides, preservatives, polymers, thickening agents, or any other ingredient commonly used in cosmetics for this type of application.

In certain embodiments, these additives are generally present in an amount ranging up to about 40% by weight of active material relative to the total weight of the composition, such as up to about 30%, up to about 20%, up to about 15%, up to about 10%, up to about 5%, such as from 0.01% to 30%.

In an embodiment, the compositions may be substantially free of reducing agents, such as thiol based reducing agents.

The compositions described herein may be in any suitable physical form. Suitable forms include, but are not limited to, low to moderate viscosity liquids, lotions, milks, gel creams, creams, pastes, clays, conditioners, masks, sprays, and the like.

If desired, a person of skill in the art can select the additives or amounts thereof in order to maintain the desired properties of the compositions. The compositions may be packaged in a variety of different containers, such as, for example, a ready-to-use container. Non-limiting examples of useful packaging include tubes, jars, caps, unit dose packages, and bottles, including squeezable tubes and bottles, bottles configured with pump dispensers, and spray bottles.

### II. Methods

Methods according to the disclosure comprise applying compositions described herein to the hair in order to treat the hair, to protect the hair from heat-induced damage, or to reduce damage to the hair. The methods can also repair hair. In an exemplary embodiment, the composition is a leave-in hair protecting composition.

The compositions may be applied to the hair before, during, or after any hair styling, shaping, caring process. In various embodiments, the compositions may be applied to wet, damp, or dry hair.

In various embodiments, the methods comprise applying a composition comprising at least one phosphate-containing compound according to disclosure to hair.

In further embodiments, the methods comprise applying a composition comprising (a) at least one compound comprising at least one phosphate, and (b) at least one amino acid, at least one hydrolyzed protein, at least one peptide or protein oligomer, or combinations thereof, to hair.

In yet further embodiments, the methods comprise (1) applying to the hair a composition comprising at least one amino acid, at least one hydrolyzed protein, at least one peptide oligomer, or a combination thereof, and (2) applying to the hair a composition comprising at least one compound comprising at least one phosphate. It should be understood that the order of applying compositions (1) and (2) is without limitation, and can be, for example, any of (i) first applying composition (1) and second applying composition (2); (ii) first applying composition (2) and second applying composition (1); or (iii) simultaneously or substantially simultaneously applying compositions (1) and (2). It should also be understood that in embodiments where compositions (1) and (2) are not applied to the hair simultaneously, it may be advantageous, but is not required, that the compositions be applied to the hair within a few minutes of each other, in order to take advantage of the synergistic effect obtained by the combination thereof. For example, compositions (1) and (2) may optionally be applied to the hair within about 5 or fewer minutes of each other, such as about 4 or fewer, about 3 or fewer, about 2 or fewer, or about 1 or fewer minutes of each other.

Although the use of heat is not required to achieve hair protection using compositions according to the disclosure, an individual may choose to heat the hair before, during, or after the composition is applied to the hair, such as, for example, using a hair dryer and/or a hair-straightening or curling device. By way of example only, the composition(s) may be applied to the hair before a heat-induced styling process, such as blow-drying; curling using a curling iron, flat iron, hot curlers, curlers with blow drying, or blowing out using a hair dryer, or straightening the hair using a flat iron or blowing out with a hair dryer.

Heating hair “at” a specified temperature means that the hair is heated with a device having the specified temperature. In various embodiments, the methods further comprise heating the hair, with a device having a temperature of up to about 250° C., such as from about 40° C. to about 210° C. In an embodiment, the hair is heated with a device having a temperature of at least about 20° C., 30° C., 40° C., 50° C., 60° C., 70° C., 80° C., 90° C., 100° C., 110° C., 120° C., 130° C., 140° C., 150° C., 160° C., 170° C., 180° C., 190° C., 200° C., 210° C., 220° C. In an embodiment, the hair is heated with a device having a temperature ranging from about 20° C. to about 220° C., such as from about 20° C. to about 130° C., about 30° C. to about 120° C., about 40° C. to about 110° C., about 50° C. to about 100° C., about 60° C. to about 90° C., about 70° C. to about 80° C., about 60° C. to about 220° C., about 70° C. to about 210° C., about 80° C. to about 200° C., about 90° C. to about 190° C., about 100° C. to about 180° C., about 110° C. to about 170° C., about 120° C. to about 160° C., or about 130° C. to about 150° C. The composition(s) may be applied to the hair before, with, or after other hair compositions (e.g., a shampoo, a conditioner, a mask, a cream, a lotion, a gel, a spray, etc.).

In various embodiments, the hair is dried or heated with a device having a temperature ranging from room temperature up to at least 40° C., for example, using a blow dryer, while optionally applying a smoothing action on the hair. The smoothing action may be conducted by brushing or combing or passing the fingers through the hair.

Drying the hair using a device having a temperature ranging from room temperature up to at least 40° C. may be accomplished by drying the hair with blow dryer device or using other heat sources such as a flat iron, a hair dryer, a heat lamp, a heat wand, or other similar devices. When a blow dryer is used, the blow dryer may be employed at a temperature ranging from about 30° C. to about 90° C. or from about 30° C. to about 60° C. or from about 35° C. to about 60° C., including ranges and sub-ranges there between.

When a flat iron is used, the flat iron may be employed at a temperature of about 100° C. or more, such as ranging from about 100° C. to about 250° C., from about 110° C. to about 230° C., from about 110° C. to about 210° C., from about 120° C. to about 200° C., from about 150° C. to about 190° C., or from about 190° C. to about 230° C., including ranges and sub-ranges there between, or at a temperature of about 230° C., about 225° C., about 220° C., about 210° C., about 200° C., about 190° C., about 180° C., about 150° C., or about 100° C., including ranges and sub-ranges there between, and preferably at about 230° C. In various embodiments, the flat iron is passed over the hair at least 1, 2, 3, 4, 5, 6, 7, 8, 9, 10 times or more, for example, up to 20, 30, 40, 50, 75, or 100 times.

Kits according to the disclosure may include two or more separately contained components. In certain embodiments, the kits comprise at least two containers comprising separately contained compositions, wherein at least one container comprises a composition comprising (a) at least one compound comprising at least one phosphate, and (b) at least one amino acid, at least one hydrolyzed protein, at least one peptide or protein oligomer, or combinations thereof. In further embodiments, the kits comprise at least two containers comprising separately contained compositions, wherein a first container comprises (1) a composition comprising at least one amino acid, at least one hydrolyzed protein, at least one peptide oligomer, or a combination thereof, and a second container comprises (2) a composition comprising at least one compound comprising at least one phosphate.

It is to be understood that although compositions and/or hair protecting processes according to the disclosure generally demonstrate one or more of the properties described herein, compositions according to the disclosure may not demonstrate all or some of the disclosed properties, yet the compositions and methods of protecting hair are still within the scope of the disclosure.

The following definitions are provided for the present disclosure only.

As used herein, the term “hair” is meant to include only hair on the scalp of the head, and does not include eyelashes or eyebrows.

The terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

The terms “a,” “an,” “the,” and “at least one” are understood to encompass the plural as well as the singular.

Thus, the term “a mixture (or combination) thereof” also relates to “mixtures (or combinations) thereof.” Throughout the disclosure, if the term “a mixture (or combination) thereof” is used, following a list of elements as shown in the following example where letters A-F represent the elements: “one or more elements selected from the group consisting of A, B, C, D, E, F, or mixtures thereof.” The term, “a mixture (or combination) thereof” does not require that the mixture (or combination) include all of A, B, C, D, E, and F (although all of A, B, C, D, E, and F may be included). Rather, it indicates that a mixture (or combination) of any two or more of A, B, C, D, E, and F can be included. In other words, it is equivalent to the phrase “one or more elements selected from the group consisting of A, B, C, D, E, F, and a mixture (or combination) of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting.

The expression “one or more” means “at least one” and thus includes individual components as well as mixtures/combinations.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” “2% to 5%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−⁷%, +/−⁶%, +/−⁵%, +/−⁴%, +/−³%, +/−²%, or +/−1%. Likewise, all endpoints of ranges are understood to be individually disclosed, such that, for example, a range of 1:2 to 2:1 is understood to disclose a ratio of both 1:2 and 2:1.

Unless otherwise indicated, all percentages herein are by weight, relative to the weight of the total composition.

The term “substantially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure.

The term “substantially free” as used herein may also mean that the specific material is not added to the composition but may still be present in a raw material that is included in the composition.

“Cosmetic composition” encompasses many types of compositions for application to hair, for example, hair lotions, hair creams, hair gel creams, hair conditioners, hair masques (masks), etc. In an embodiment, the compositions are leave-on hair compositions.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

### EXAMPLES

The following examples are intended to be non-limiting and explanatory in nature only.

### Example 1

Swatch studies were performed on previously (approximately 1 week prior) double bleached swatches of hair to evaluate the impact of the disclosed compositions on the denaturation temperature of hair as measured by Differential Scanning calorimetry (DSC).

Double bleached hair swatches were washed, dabbed dry, and each treated with one of the exemplified compositions in Example 1 by applying 0.4 g of one of the compositions disclosed in this Example/gram of hair with a pipette and working it in with gloved hands and a comb. After waiting 5 minutes, each swatch was roughly blow dried for 120 seconds then brush blow dried 10 passes. For swatches that were blow-dried and flat ironed, each blow-dried swatch was separated into two halves, and one of each of the halves was flat-ironed for 3 passes at 190° C. About 8 mg of finely cut hair taken from the middle portion of the swatch (not including tip and root sections) was subjected to DSC. DSC was then performed on cut hair from each swatch with 50 μL water added to a sealed pan. The sealed pan containing the hair mixed in water sat overnight at room temperature (˜16 hours) before being run in the DSC instrument. The samples were heated at a ramp rate of 10° C./min to 180° C. The data was analyzed to determine the denaturation temperature (Td) of each swatch. A higher Td value means that more heat is required to denature the keratin protein.

### Set 1

The following hair protecting compositions were prepared by mixing the components, as disclosed in Table 1. All the compositions had a pH of 7.5 (pH adjusted as necessary with sodium hydroxide or lactic acid). Concentrations of Compositions 1A, 1B, and 1D were selected to achieve a 1:1 molar ratio between the compositions (i.e., a 1:1:1 molar ratio of the single phosphate in MSP, the single phosphate in glycerol phosphate (disodium glycerol phosphate), and the six phosphates in phytic acid was maintained). Concentrations of Compositions 1A, 1B, and 1C were selected to achieve 1:1 ratio of phosphate functional groups between the compositions (i.e., a 1:1:1 ratio of the single phosphate in MSP, the single phosphate in glycerol phosphate (disodium glycerol phosphate), and one phosphate in phytic acid was maintained). Amounts are expressed in wt % of active materials:

TABLE 1

| | Composition Set 1 | | | | |
|---|---|---|---|---|---|
| | 1A | 1B | 1C | 1D | 1E |
| Monosodium | 0.8% | — | — | — | — |
| Phosphate | | | | | |
| Glycerol | — | 1.44% | — | — | — |
| phosphate | | | | | |
| Phytic Acid | — | — | 0.73% | 4.4% | — |
| DI water | Q.S. 100 | Q.S. 100 | Q.S. 100 | Q.S. 100 | Q.S. 100 |

Compositions 1A to 1E were applied to separate double bleached hair swatches as described above. The Td temperature for each swatch was determined to be as follows:

TABLE 1b

| | BD* | | BD + FI* | |
|---|---|---|---|---|
| | Td (° C) | SD* | Td (° C) | SD |
| 1E (first**): DI H2O pH 7.5 | 130.2 | 0.2 | 129.4 | 0.5 |
| 1A: MSP 0.8% | 134.9 | 0.2 | 132.6 | 0.2 |
| 1E (second**): DI H2O pH 7.5 | 128.3 | 0.3 | 128.3 | 0.6 |
| 1B: Glycerol Phosphate 1.44% | 130.9 | 0.3 | 130.7 | 0.4 |
| 1C: Phytic Acid 0.73% | 134.3 | 0.6 | 133.9 | 0.2 |
| 1D: Phytic Acid 4.4% | 137.1 | 0.3 | 134.6 | 0.1 |

*In the Examples, “BD” refers to blow out, “FI” refers to flat iron, and “SD” refers to standard deviation. **In the Examples, “first” and “second” for the referenced compositions refers to the first and second bar in the respective Figure.

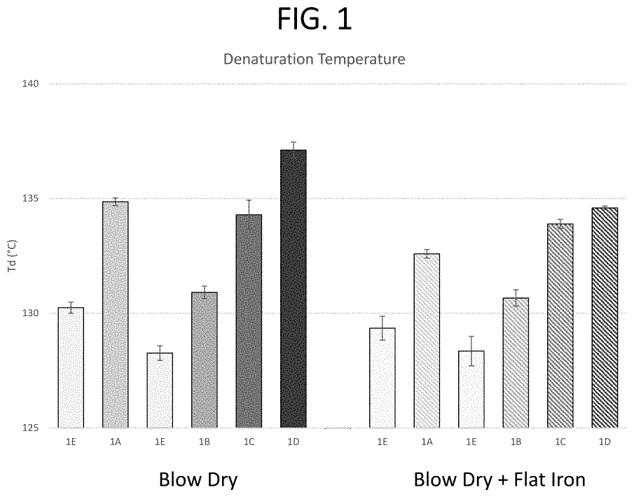

All the phosphate containing compositions (1A-1D) imparted a statistically significant increase in hair Td after blow drying and after blow drying+flat ironing. As shown in FIG. 1, the relative increase (improvement) in Td values imparted by each composition (both for the blow dried and blow dried+flat ironed) was determined to be as follows: 1D (phytic acid 4.4%) showing the best improvement in Td>1C (phytic acid 0.73%)>1A (MSP 0.8%)>1B (glycerol phosphate 1.44%)>1E (DI water). There was no statistical difference in Td values between blow drying and blow drying+flat ironing for 1B (glycerol phosphate) and 1C (phytic acid 0.73%); but these Td values were still higher than the Td values for water. On the other hand, the additional flat ironing resulted in a decrease in Td values for 1A (MSP) and 1D (phytic acid 4.4%).

The compositions comprising phosphates surprisingly demonstrated beneficial heat protecting properties, thus better protecting the hair from heat. Blow drying, with or without flat-ironing, further protected the hair from damage incurred in subsequent heat treatments for all the phosphate containing compositions as compared to compositions that did not contain phosphates (i.e. DI water only).

### Set 2

The following hair protecting compositions were prepared by mixing the components, as disclosed in Table 2. The compositions were pH adjusted as necessary with sodium hydroxide or hydrochloric acid. Amounts are expressed in wt % of active materials:

TABLE 2

| | Composition Set 2 | | | | | |
|---|---|---|---|---|---|---|
| | 2A | 2B | 2C | 20 | 2E | 2F |
| Monosodium | — | — | — | 0.8% | 0.8% | 0.8% |
| Phosphate | | | | | | |
| pH | 2.5 | 5.9 | 7.5 | 2.5 | 4.8 | 7.5 |
| | (pH | (not | (pH | (pH | (not | (pH |
| | adjusted) | adjusted) | adjusted) | adjusted) | adjusted) | adjusted) |
| DI | Q.S. 100 | Q.S. 100 | Q.S. 100 | Q.S. 100 | Q.S. 100 | Q.S. 100 |
| (deionized) | | | | | | |
| water | | | | | | |

Compositions 2A to 2F were applied to separate double bleached hair swatches as described above. The Td temperature for each swatch was determined to be as follows:

TABLE 2b

| | BD | | BD + FI | |
|---|---|---|---|---|
| | Td (° C) | SD | Td (° C) | SD |
| 2A: DI H2O pH 2.5 | 128.5 | 1.2 | 128.7 | 1.1 |
| 2B: DI H2O pH 5.9 | 130.2 | 0.6 | 128.3 | 0.8 |
| 2C: DI H2O pH 7.5 | 129.7 | 0.7 | 130.1 | 0.2 |
| 20: MSP 0.8% pH 2.5 | 134.1 | 1.0 | 133.8 | 1.1 |
| 2E: MSP 0.8% pH 4.8 | 133.7 | 2.1 | 133.2 | 1.1 |
| 2F: MSP 0.8% pH 7.5 | 133.8 | 1.4 | 132.7 | 0.8 |

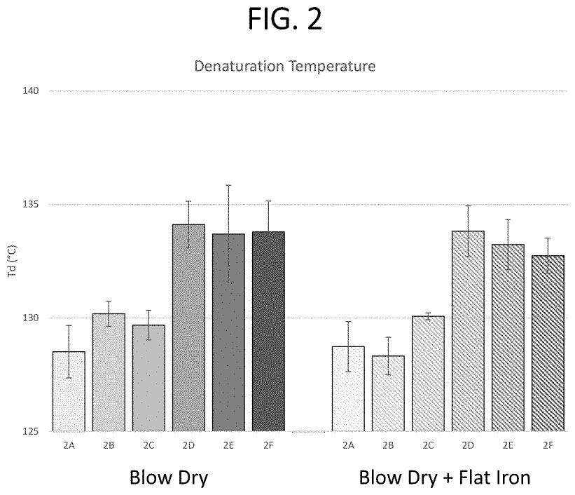

As shown in FIG. 2, the MSP treatment at all three pH values (compositions 2D-2F) resulted in a statistically significant increase in hair Td compared to the DI water treatment at similar pH values for both the blow dried and the blow dried+flat ironed swatches. There was no statistical difference in Td values between the swatches treated with MSP at different pH values or swatches treated with MSP samples then blow dried and flat ironed, indicating that the composition's performance is pH independent. The Td values for the blow dried hair ranked as follows: MSP 0.8% pH 2.5˜MSP 0.8% pH 7.5˜MSP 0.8% pH 4.8>DI water pH 5.9˜DI water pH 7.5˜DI water pH 2.5. And the Td values for the blow dried+flat ironed hair ranked as follows: MSP 0.8% pH 2.5˜MSP 0.8% pH 4.8˜MSP 0.8% pH 7.5>DI water pH 7.5˜DI water pH 2.5˜DI water pH 5.9. (“˜” meaning “approximately equal” or “similar” throughout the Examples.)

The compositions comprising phosphates surprisingly demonstrated beneficial heat protecting properties, thus better protecting the hair from heat, irrespective of the composition's pH.

### Set 3

The following hair protecting compositions were prepared by mixing the components, as disclosed in Table 3. All the compositions had a pH of 7.5 (pH adjusted as necessary with NaOH). Amounts are expressed in wt % of active materials:

TABLE 3

| | Composition Set 3 | | | |
|---|---|---|---|---|
| | 3A | 3B | 3C | 3D |
| Monosodium | — | 0.4% | 0.8% | 1.6% |
| Phosphate | | | | |
| DI water | Q.S. 100 | Q.S. 100 | Q.S. 100 | Q.S. 100 |

Compositions 3A to 3D were applied to separate double bleached hair swatches as described above. The Td temperature for each swatch was determined to be as follows:

TABLE 3b

| | BD | | BD + FI | |
|---|---|---|---|---|
| | Td (° C) | SD | Td (° C) | SD |
| 3A: DI H2O pH 7.5 | 130.2 | 0.2 | 129.4 | 0.5 |
| 3B: MSP 0.4% | 130.8 | 0.3 | 131.7 | 0.1 |
| 3C: MSP 0.8% | 134.9 | 0.2 | 132.6 | 0.2 |
| 3D: MSP 1.6% | 134.0 | 0.3 | 135.5 | 0.1 |

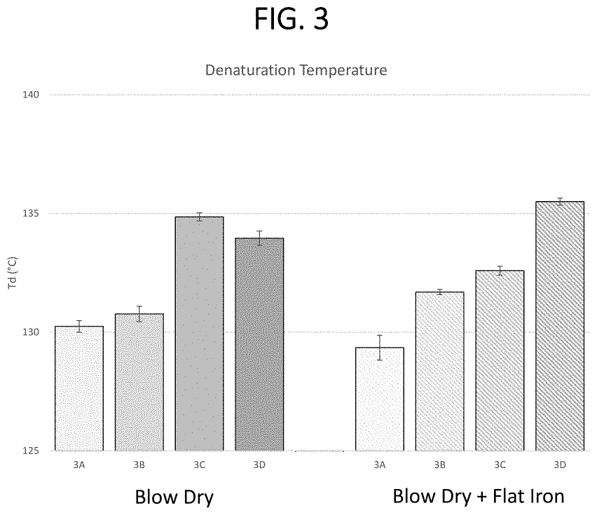

As shown in FIG. 3, compositions 3B-3D statistically increased the Td of the hair swatches compared to DI water treatment (3A) for both for the blow dried swatches and the blow dried+flat ironed swatches. For the blow dried+flat ironed swatches, the Td increased with increasing concentration of applied MSP. For the blow dried swatches, the Td increased with increasing concentration of MSP, except the swatch treated with composition 3C (MSP 1.6%) had a lower Td than the swatch treated with composition 3D (MSP 0.8%). Thus, the Td values for the blow dried hair ranked as follows: MSP 0.8%>MSP 1.6%>MSP 0.4%>DI water. And the Td values for the blow dried and flat ironed hair ranked as follows: MSP 1.6%>MSP 0.8%>MSP 0.4%>DI water.

The compositions comprising phosphates surprisingly demonstrated beneficial heat protecting properties over a range of concentrations, thus better protecting the hair from heat.

### Set 4

The following hair protecting compositions were prepared by mixing the components, as disclosed in Table 4. All the compositions had a pH of 7.5 (pH adjusted as necessary with lactic acid and sodium hydroxide. Concentrations were selected to maintain 1:1 molar ratio between the molecules in each composition. Amounts are expressed in wt % of active materials:

TABLE 4

| | Composition Set 4 | | | | |
|---|---|---|---|---|---|
| | 4A | 4B | 4C | 4D | 4E |
| Monosodium | − | 0.8% | − | − | − |
| Phosphate | | | | | |
| Lysine | − | − | 0.97% | − | − |
| Threonine | − | − | − | 0.8% | − |
| Aspartic Acid | − | − | − | − | 0.89% |
| DI water | Q.S. 100 | Q.S. 100 | Q.S. 100 | Q.S. 100 | Q.S. 100 |

Compositions 4A to 4E were applied to separate double bleached hair swatches as described above. In addition, compositions 4C to 4E (containing 0.8-0.97% amino acid) were each applied to one of an additional three swatches in a first step, and composition 4B (containing 0.8% MSP) was applied to all 3 swatches in a second consecutive step (applying 0.4 g/gram of hair of composition and waiting 5 min for each step). The Td temperature for each swatch was determined to be as follows:

TABLE 4b

| | BD | | BD + FI | |
|---|---|---|---|---|
| | Td (° C) | SD | Td (° C) | SD |
| 4A: DI H20 pH 7.5 | 131.3 | 0.4 | 132.2 | 0.2 |
| 4B: MSP 0.8% | 134.7 | 0.3 | 134.8 | 0.2 |
| 4C: Lysine 0.97% | 134.3 | 0.4 | 132.5 | 0.3 |
| 4D: Threonine 0.8% | 132.8 | 0.1 | 132.1 | 0.3 |
| 4E: Aspartic Acid 0.89% | 134.0 | 0.3 | 133.8 | 0.3 |
| 4C + 4B: Lys 0.97% | 136.5 | 0.2 | 137.2 | 0.3 |
| MSP 0.8% 2 Step | | | | |
| 4C +4B: Thr 0.8% + | 136.1 | 0.3 | 135.2 | 0.5 |
| MSP 0.8% 2 Step | | | | |
| 4E + 4B: Asp Ac | 138.4 | 0.3 | 136.6 | 0.3 |
| 0.89% + MSP | | | | |
| 0.8% 2 Step | | | | |

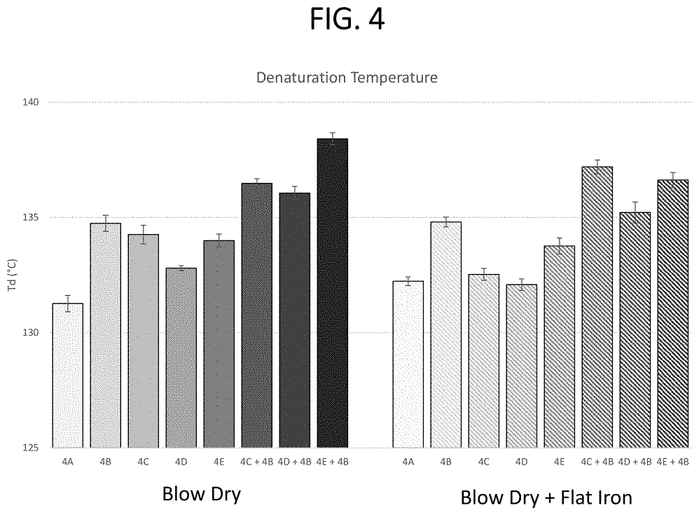

As shown in FIG. 4, the application of MSP (4B) and any of the three amino acids (4C to 4E) increases the Td of blow-dried hair, in a statistically significant manner, compared to the application of MSP alone (4B). The application of MSP (4B) and aspartic acid or lysine (4C or 4E) increases the Td of blow-dried and flat-ironed hair, in a statistically significant manner, compared to the application of MSP alone (4B). Thus, the Td values for the blow dried hair ranked as follows: aspartic acid+MSP>lysine+MSP threonine+MSP>MSP lysine aspartic acid>threonine>DI water. The Td values for the blow dried+flat ironed hair ranked as follows: lysine+MSP>aspartic acid+MSP>threonine+MSP MSP>aspartic acid>lysine˜DI water˜threonine. The compositions comprising amino acids surprisingly demonstrated beneficial heat protecting properties, thus better protecting the hair from heat, irrespective of the type of amino acid used.

### Set 5

The following hair protecting compositions were prepared by mixing the components, as disclosed in Table 5. All the compositions had a pH of 7.5 (pH adjusted as necessary with sodium hydroxide). Amounts are expressed in wt % of active materials:

| | Composition Set 5 | | |
|---|---|---|---|
| | 5A | 5B | 5C |
| Monosodium | − | 0.8% | − |
| Phosphate | | | |
| Hydrolyzed | − | − | 0.8% |
| Soy Protein | | | |
| DI water | Q.S. 100 | Q.S. 100 | Q.S. 100 |

The hydrolyzed soy protein comprised primarily low molecular weight hydrolyzed protein: 79.4% of the protein was less than 1000 g/mol and 98.5% was less than 2990 g/mol. Compositions 5A to 5C were applied to separate double bleached hair swatches as described above in Example 1. In addition, composition 5C (containing 0.8% hydrolyzed protein) was applied to an additional swatch as a first step, and composition 5B (containing 0.8% MSP) was applied to the swatch in a second consecutive step (applying 0.4 g/gram of hair of composition and waiting 5 min for each step). applied to separate double bleached hair swatches as described above. The Td temperature for each swatch was determined to be as follows:

TABLE 5b

| | BD | | BD + FI | |
|---|---|---|---|---|
| | Td (° C) | SD | Td (° C) | SD |
| 5A: DI H2O pH 7.5 | 131.3 | 0.4 | 132.2 | 0.2 |
| 5B: MSP 0.8% | 134.7 | 0.3 | 134.8 | 0.2 |
| 5C: HYDROLYZED | 133.4 | 0.8 | 133.8 | 0.3 |
| SOY PROTEIN 0.8% | | | | |
| 5B + 5C: | 135.1 | 0.3 | 136.2 | 0.1 |
| HYDROLYZED | | | | |
| SOY PROTEIN | | | | |
| 0.8% + MSP 0.8% | | | | |

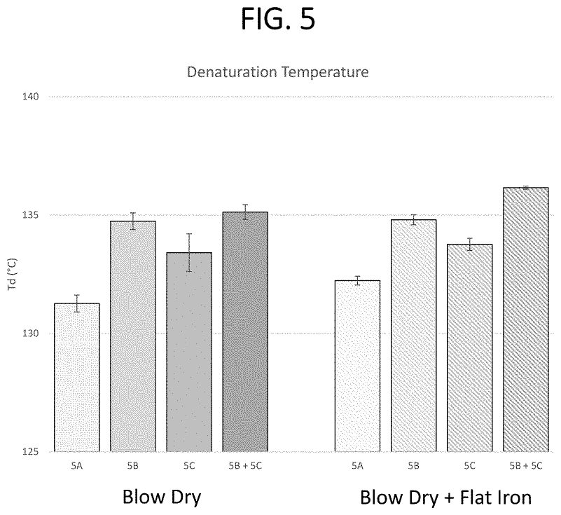

As shown in FIG. 5, hydrolyzed protein in combination with MSP statistically increased the Td above the controls of the blow-dried+flat ironed swatches. Hydrolyzed protein in combination with MSP statistically increased the Td above the DI water (5A) and hydrolyzed protein only (5C) controls, but did not statistically improve the Td above the MSP control (5B) for the blow dry only samples. The application of MSP (5B) and hydrolyzed protein (5C) increases the Td of blow-dried+flat-ironed hair, in a statistically significant manner, compared to the application of MSP alone (5B). Thus, the Td values for the blow dried hair ranked as follows: hydrolyzed protein+MSP MSP>hydrolyzed protein>DI water. The Td values for the blow dried hair and flat-ironed ranked as follows: hydrolyzed protein+MSP>MSP>hydrolyzed protein>DI water.

The compositions comprising hydrolyzed protein surprisingly demonstrated beneficial heat protecting properties alone or synergistically in combination with phosphates, thus better protecting the hair from heat.

### Example 2

Swatch studies were performed on natural hair swatches, double bleached hair swatches treated immediately after bleaching (“Bleached Fresh”), and double bleached hair swatches treated approximately one week after bleaching (“Bleached 1 Week”) to evaluate the impact of the disclosed compositions on the denaturation temperature of hair as measured by Differential Scanning calorimetry (DSC).

The following compositions were prepared by mixing the components, as disclosed in Table 6. All the compositions had a pH of 7.5 (pH adjusted as necessary with NaOH). Amounts are expressed in wt % of active materials:

TABLE 6

| | Composition Set 6 | |
|---|---|---|
| | 6A | 6B |
| Monosodium | − | 0.8% |
| Phosphate | | |
| DI water | Q.S. 100 | Q.S. 100 |

Three sets of hair swatches (natural hair swatches, double bleached hair swatches immediately after bleaching, and double bleached hair swatches approximately one week after bleaching) were washed, dabbed dry, and separately treated with one of the exemplified compositions in Example 2 by applying 0.4 g of the composition/gram of hair with a pipette and working it in with gloved hands and a comb. After waiting 5 minutes, each swatch was rough blow dried for 120 seconds then brush blow dried 10 passes. The swatch was then flat ironed for 3 passes at 190° C. About 8 mg of finely cut hair taken from the middle portion of the swatch (not including tip and root sections) was used. DSC was then performed on cut hair from each swatch with 50 μL water added to the sealed pan. The samples were heated at a ramp rate of 10° C./min to 180° C. The data was analyzed to determine the denaturation temperature (Td) of each swatch:

| | BD | | BD + FI | |
|---|---|---|---|---|
| | Td (° C) | SD | Td (° C) | SD |
| 6A + Natural Hair: | 153.6 | 0.1 | 153.0 | 0.8 |
| DI H2O pH 7.5 | | | | |
| 6B + Natural Hair: | 154.7 | 0.3 | 154.8 | 0.1 |
| MSP 0.8% | | | | |
| 6A + 1 Week Bleached Hair: | 131.3 | 0.4 | 132.2 | 0.2 |
| DI H2O pH 7.5 2x bleach | | | | |
| 6B + 1 Week Bleached Hair: | 134.7 | 0.3 | 134.8 | 0.2 |
| MSP 0.8% 2x bleach | | | | |
| 6A + Fresh Bleached Hair: | 129.7 | 0.4 | 128.9 | 0.4 |
| DI H2O pH 7.5 Fresh Bleach | | | | |
| 6B + Fresh Bleached Hair: | 131.6 | 0.2 | 131.4 | 0.4 |
| MSP 0.8% Fresh Bleach | | | | |

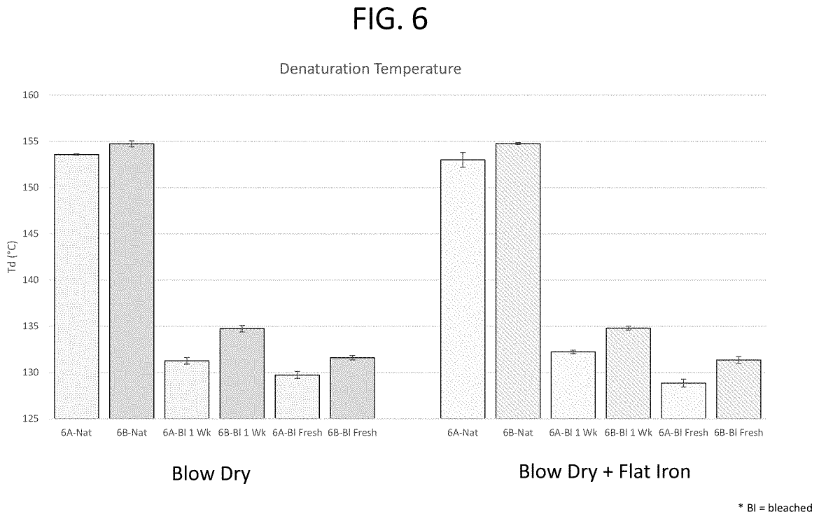

As shown in FIG. 6, Composition 6A comprising MSP statistically increased the Td of natural hair, freshly bleached hair, and previously bleached hair. There was no difference in Td values between the blow dry and blow dry+flat iron samples for each type of hair when treated with composition 6A. As also shown in FIG. 6, the Td varied greatly by the hair type with Td for natural hair being the highest, and Td for freshly bleached hair the lowest. The percent change in Td values between hair treated with MSP 0.8% and hair treated with water alone was highest for previously bleached hair and lowest for natural hair.

The compositions comprising phosphates surprisingly demonstrated beneficial heat protecting properties when applied to different types of chemically treated hair, thus better protecting the hair from heat.

### Example 3

Swatch studies were performed on double bleached hair to evaluate the impact of the disclosed compositions on the denaturation temperature of hair as measured by Differential Scanning calorimetry (DSC).

The following compositions were prepared by mixing the components, as disclosed in Table 7. All the compositions had a pH of 7.5 (pH adjusted as necessary with NaOH). Amounts are expressed in wt % of active materials:

TABLE 7

| | Composition Set 7 | |
|---|---|---|
| | 7A | 7B |
| Monosodium | − | 0.8% |
| Phosphate | | |
| DI water | Q.S. 100 | Q.S. 100 |

Double bleached hair swatches were washed, dabbed dry, and separately treated with one of the exemplified compositions in Example 3 by applying 0.4 g of the composition/gram of hair with a pipette and working it in with gloved hands and a comb and waiting for 5 min.

The blow-dried swatches were rough blow dried for 120 seconds then brush blow dried 10 passes. The flat ironed swatches were additionally flat ironed for 3 passes at 190° C. Control swatches were treated with compositions 7A or 7B, air dried overnight at ambient temperature, and were not treated with any heat. About 8 mg of finely cut hair taken from the middle portion of the swatch (not including tip and root sections) was used. About 8 mg of finely cut hair taken from the middle portion of the swatch (not including tip and root sections) was used. DSC was then performed on cut hair from each swatch with 50 μL water added to the sealed pan. The samples were heated at a ramp rate of 10° C./min to 180° C. The data was analyzed to determine the denaturation temperature (Td) of each swatch:

TABLE 7b

| | Td | SD |
|---|---|---|
| 7A (Air Dry): DI H2O pH 7.5 | 131.4 | 0.4 |
| 7A (BD): DI H2O pH 7.5 | 129.7 | 0.7 |
| 7A (BD + Fl): DI H2O pH 7.5 | 130.1 | 0.2 |
| 7B (Air Dry): MSP 0.8% pH 7.5 | 132.8 | 0.6 |
| 7B (BD): MSP 0.8% pH 7.5 | 133.8 | 1.4 |
| 7B (BD + Fl): MSP 0.8% pH 7.5 | 132.7 | 0.8 |

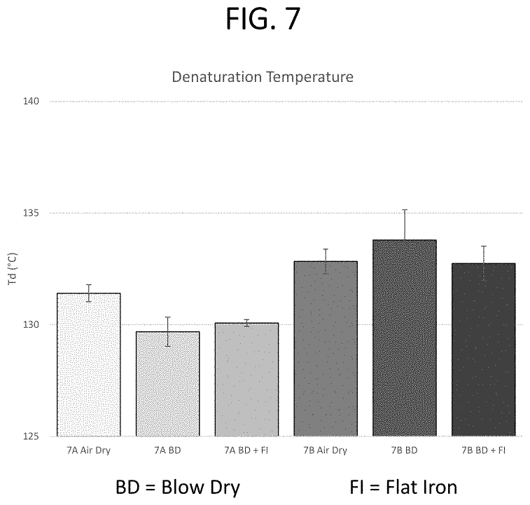

As shown in FIG. 7, the MSP treatment applied to the hair in all three treatment methods (composition 7B) resulted in a statistically significant increase in Td compared to the DI water using similar treatment methods (either air dried, blow dried, or blow dried+flat ironed). There was no statistical difference in Td values between the MSP samples, indicating that performance is heat independent. Thus, the Td values for the various methods ranked as follows: MSP 0.8% BD MSP 0.8% Air Dry˜MSP 0.8% FI>DI water Air Dry>DI water FI˜DI water BD.

The compositions comprising phosphates surprisingly demonstrated beneficial heat protecting properties when applied to air dried or heat treated hair, thus better protecting the hair from heat.

### Example 4

Swatch studies were performed on natural hair to evaluate the impact of the disclosed compositions on the denaturation temperature of hair as measured by Differential Scanning calorimetry (DSC).

The following hair protecting compositions were prepared by mixing the components, as disclosed in Table 8. All the compositions had a pH of 7.5 (pH adjusted as necessary with NaOH or lactic acid), except the pH of the DI water was not pH adjusted. Amounts are expressed in wt % of active materials:

TABLE 8

| | Composition Set 8 | | | |
|---|---|---|---|---|
| | 8A | 8B | 8C | 8E |
| Monosodium | − | 0.8% | − | − |
| Phosphate | | | | |
| Glycerol | − | − | 0.8% | − |
| phosphate | | | | |
| Phytic Acid | − | − | − | 0.4% |
| DI water | Q.S. 100 | Q.S. 100 | Q.S. 100 | Q.S. 100 |

Natural hair swatches were washed, dabbed dry, and separately treated with one of the exemplified compositions in Example 4 by applying 0.2 g of the composition/gram of hair with a pipette and working it in with gloved hands and a comb for 1 min then waiting for 3 min.

The swatches were rough blow dried until dry (about 10 minutes). The swatches were then flat ironed for 10 passes at 232° C. at a rate of 10 seconds/stroke. Control swatches were treated with DI water that was not pH adjusted (composition 8A) and were not treated with any heat. The wash, composition application, and heat cycle were repeated two more times for a total of 3 applications and heat cycles. Swatches were then washed with and dialyzed for 16 hours in DI water followed by air drying.

About 8 mg of finely cut hair taken from the middle portion of the swatch (not including tip and root sections) was used. DSC was then performed on cut hair from each swatch with 50 μL water added to the sealed pan. The samples were heated at a ramp rate of 10° C./min to 180° C. The data was analyzed to determine the denaturation temperature (Td) of each swatch.

TABLE 8b

| | Td (° C) | SD |
|---|---|---|
| 8A (first): DI Water + Air Dry | 155.4 | 0.4 |
| 8A (first): DI Water + BD | 148.9 | 0.3 |
| 8B: MSP 0.8% + BD | 151.2 | 0.3 |
| 8A (second): DI Water + Air Dry | 153.9 | 0.7 |
| 8A (second): DI Water + BD | 149.5 | 0.9 |
| 8C: Glycerol Phosphate 0.8% + BD | 150.9 | 0.2 |
| 8E: Phytic Acid 0.4% + BD | 150.5 | 0.1 |

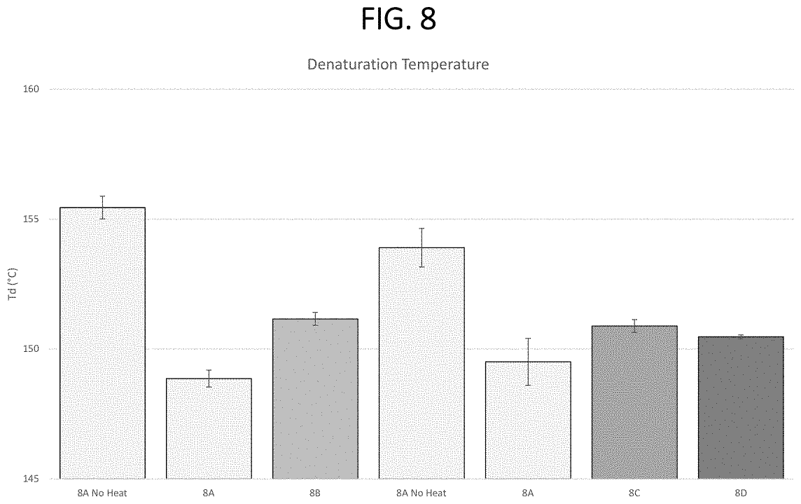

As shown in FIG. 8, the phosphate-comprising compositions applied to the hair in all three treatment methods (compositions 8B, 8C, and 8E) resulted in a statistically significant increase in Td of hair subjected to heat, compared to hair treated with DI water using similar treatment methods with heat. There was no statistical difference in Td values between the MSP and glycerol phosphate samples. MSP 0.8% and glycerol phosphate 0.8% performed better than phytic acid 0.4% in a statistically significant manner. Thus, the Td values for the various methods ranked as follows: MSP 0.8% BD˜glycerol phosphate 0.8% BD>phytic acid 0.4% BD>DI water BD.

The compositions comprising phosphates surprisingly demonstrated beneficial heat protecting properties when applied to heat treated hair, thus better protecting the hair from heat.

1-66. (canceled)

67. A hair protecting composition comprising at least one compound comprising at least one phosphate group, wherein the pH of the composition is less than or equal to about 7.5.

68. The hair protecting composition of claim 67, wherein the at least one compound comprising at least one phosphate group is present in an amount of up to about 10%, based on the total weight of the composition.

69. The hair protecting composition of claim 67, wherein the at least one compound comprising at least one phosphate group is chosen from monosodium phosphate, phytic acid, glycerol phosphate, or mixtures thereof.

70. The hair protecting composition of claim 67, wherein the at least one compound comprising at least one phosphate group comprises monosodium phosphate, and wherein the monosodium phosphate is present in an amount ranging from about 0.001% to about 2%, relative to the total weight of the composition.

71. The hair protecting composition of claim 67, wherein the at least one compound comprising at least one phosphate group comprises glycerol phosphate, and wherein the glycerol phosphate is present in an amount ranging from about 0.001% to about 10%, relative to the total weight of the composition.

72. The hair protecting composition of claim 67, wherein the at least one compound comprising at least one phosphate group comprises phytic acid, and wherein the phytic acid is present in an amount ranging from about 0.001% to about 10%, relative to the total weight of the composition.

73. The hair protecting composition of claim 67, wherein the composition further comprises at least one amino acid, at least one hydrolyzed protein, at least one peptide oligomer, at least one protein oligomer, or combinations thereof.

74. The hair protecting composition of claim 73, wherein at least one amino acid, at least one hydrolyzed protein, at least one peptide oligomer, at least one protein oligomer, or combinations thereof is present in an amount of up to about 5%, based on the total weight of the composition.

75. The hair protecting composition of claim 67, further comprising at least one amino acid chosen from lysine, threonine, aspartic acid, or combinations thereof.

76. The hair protecting composition of claim 67, further comprising at least one hydrolyzed protein.

77. The hair protecting composition of claim 73, wherein the weight ratio of the total amount of the at least one compound comprising at least one phosphate to the total amount of the at least one amino acid, at least one hydrolyzed protein, at least one peptide oligomer, at least one protein oligomer, or combinations thereof ranges from about 1:4 to about 4:1.

78. A method for protecting hair or reducing heat-induced damage to hair, said method comprising applying to the hair a composition comprising at least one compound comprising at least one phosphate, wherein the pH of the composition is less than or equal to about 7.5.

79. The method of claim 78, wherein the method further comprises a second step comprising heating the hair at a temperature ranging from about 40° C. to about 210° C., wherein the second step occurs after applying the composition to the hair.

80. The method of claim 78, wherein the at least one compound comprising at least one phosphate is present in an amount of up to about 10%, based on the total weight of the composition.

81. The method of claim 78, wherein the at least one compound comprising at least one phosphate is chosen from monosodium phosphate, phytic acid, glycerol phosphate, or combinations thereof.

82. The method of claim 78, wherein the composition further comprises at least one amino acid, at least one hydrolyzed protein, at least one peptide oligomer, at least one protein oligomer, or combinations thereof.

83. The method of claim 82, wherein at least one amino acid, at least one hydrolyzed protein, at least one peptide oligomer, at least one protein oligomer, or combination thereof is present in an amount of up to about 5%, based on the total weight of the composition.

84. The method of claim 78, wherein the composition further comprises at least one hydrolyzed protein.

85. The method of claim 82, wherein the weight ratio of the total amount of the at least one compound comprising at least one phosphate to the total amount of the amino acid, at least one hydrolyzed protein, at least one peptide oligomer, at least one protein oligomer, or combinations thereof ranges from about 1:4 to about 4:1.

86. A kit comprising at least two containers, wherein the first container comprises (1) a composition comprising at least one amino acid, at least one hydrolyzed protein, at least one peptide oligomer, at least one protein oligomer, or a combination thereof; and the second container comprises (2) a composition comprising at least one compound having at least one phosphate group.
